(19) United States
(12) Patent Application Publication
(10) Pub. No.: US 2008/0038712 A1
(43) Pub. Date: Feb. 14, 2008

(54) Oligomer Probe Array with Improved Signal-to-Noise Ratio and Detection Sensitivity and Method of Manufacturing the Same

(76) Inventors: Jung-hwan Hah, Hwaseong-si (KR); Sung-min Chi, Hwaseongi-si (KR); Kyoung-seon Kim, Suwon-si (KR); Won-sun Kim, Suwon-si (KR)

Correspondence Address: F. CHAU & ASSOCIATES, LLC, 130 WOODBURY ROAD, WOODBURY, NY, 11797, US

(21) Appl. No.: 11/686,492

(22) Filed: Mar. 15, 2007

(30) Foreign Application Priority Data
May 2, 2006 (KR) 10-2006-0039713

(51) Int. Cl. G01N 33/53

(52) U.S. Cl. 435/4

(57) ABSTRACT

An oligomer probe array with improved signal-to-noise ratio and desired detection sensitivity ever when a reduced design rule is employed includes a substrate, a plurality of probe cell active regions formed on or in the substrate, each of the plurality of probe cell active regions having a three-dimensional surface and being coupled, with at least one oligomer probe with its own sequence, and a probe cell isolation region defining the probe cell active regions and having no functional groups for coupling with the oligomer probes on a surface.

This application claims priority from Korean Patent Application No. 10-2006-0039713 filed on May 2, 2006 in the Korean Intellectual Property Office, the contents of which are incorporated herein by reference in their entirety.

### BACKGROUND OF THE INVENTION

1. Field of the Invention

The present disclosure is directed to an oligomer probe array, and more particularly, to an oligomer probe array with improved Signal-to-Noise Ratio (hereinafter, referred to as “SNR”) and detection sensitivity, and methods of manufacturing the same.

2. Description of the Related Art

An oligomer probe array is a tool that has been widely used in gene expression profiling, genotyping, detection of mutations or polymorphisms such as Single-Nucleotide Polymorphism (SNP), protein or peptide assays, potential drug screening, development and preparation of novel drugs, etc.

Currently widely available oligomer probe arrays include a plurality of probe cell arrays manufactured by activating predetermined regions of a substrate using light (e.g., UV) irradiation followed by in-situ synthesis of oligomer probes in the photo-activated regions.

However, when repeating a photolithography process for the in-situ synthesis of oligomer probes, mask misalignment, may be caused or stray light may arise from diffracted light, thereby leading to activation of some undesired regions of a substrate, and thus, formation of oligomer byproducts in the undesired regions. Such non-specific oligomer formation causes a low SNR in data analysis for hybridization of a target sample with oligomer probes, which renders accurate data analysis difficult.

Meanwhile, as oligomer probe array-based analysis is shifted down to the nucleotide (minimal unit of DNA) level from the gene level, the design rule of probe cells is reduced beyond several tens of μm to several μm. Thus, the effect of SNR on accuracy of data analysis is being significantly increased.

In oligonucleotide (“oligomer”) probe arrays that are currently available, in order to guarantee minimum detection sensitivity, 0.01-1 femtomole of oligonucleotide probes are coupled to each photo-activated region of 10-100/μm. However, if the design rule of a probe cell is reduced to less than 1 μm, the spacing between oligonucleotide probes is about 4 nm, and thus, a small quantity (about 0.1 attomoles) of oligonucleotide probes is present in each photo-activated region. The use of such a small quantity of oligonucleotide probes makes it difficult to secure absolute minimum detection sensitivity required for analysis.

### SUMMARY OF THE INVENTION

According to at least one exemplary embodiment of the present invention, an oligomer probe array includes a substrate, a plurality of probe cell active regions formed on or in the substrate, each of the plurality of probe cell active regions having a three-dimensional surface and being coupled with at least one oligomer probe with its own sequence, and a probe cell isolation region defining the probe cell active regions and having no functional groups for coupling with the oligomer probes on a surface.

According to at least one exemplary embodiment of the present invention, a method of manufacturing an oligomer probe array includes providing a substrate, forming a plurality of probe cell active regions with a three-dimensional surface on or in the substrate, the plurality of probe cell active region being defined by a probe cell isolation region without functional groups for coupling with oligomer probes and coupling the oligomer probes to the plurality of probe cell active regions such that each of the probe cell active regions is coupled with at least one oligmer probe with Its own sequence.

### BRIEF DESCRIPTION OF THE DRAWINGS

The above and other features of the present invention will become more apparent by describing in detail exemplary embodiments thereof with reference to the attached drawings.

FIGS. 1A and 1B are layouts of probe cell active regions of oligomer probe arrays according to at least one embodiment of the present invention.

FIGS. 2 through 5 are sectional views illustrating oligomer probe arrays including a plurality of probe cell active regions having a three-dimensional surface on a substrate, according to at least one embodiment of the present invention.

FIGS. 6 through 9 are sectional views illustrating oligomer probe arrays including a plurality of probe cell active regions having a three-dimensional surface formed from LOCOS (LOCal Oxidation of Silicon) oxide layers formed by local oxidation of a substrate, according to another embodiment of the present invention.

FIGS. 10 through 13 are sectional views illustrating oligomer probe arrays including a plurality of trench-type probe cell active regions having a three-dimensional surface in a substrate, according to a further embodiment of the present invention.

FIGS. 14 through 17 are sectional views of intermediate structures illustrating a method of manufacturing an oligomer probe array as illustrated in FIG. 2.

FIGS. 18 and 19 are sectional views of intermediate structures illustrating another method of manufacturing an oligomer probe array as illustrated in FIG 2.

FIGS. 20 through 23 are sectional views of intermediate structures illustrating a method of manufacturing an oligomer probe array as illustrated in FIG. 3.

FIG. 24 is a sectional view of an intermediate structure illustrating a method of manufacturing an oligomer probe array as illustrated in FIG. 4

FIG. 25 is a sectional view of an intermediate structure illustrating a method of manufacturing an oligomer probe array as illustrated in FIG. 5

FIGS. 26 and 27 are sectional views of intermediate structures illustrating a method of manufacturing an oligomer probe array as illustrated in FIG. 6

FIGS. 28 and 29 are sectional views of intermediate structures illustrating a method of manufacturing an oligomer probe array as illustrated in FIG. 10.

### DETAILED DESCRIPTION OF THE EXEMPLARY EMBODIMENTS

Features of embodiments of the invention and methods of accomplishing the same may be understood more readily by reference to the following detailed description of exemplary embodiments and the accompanying drawings. Embodiments of the invention may, however, be embodied in many different forms and should not be construed as being limited to the embodiments set forth herein, in the accompanying drawing figures, common reference numbers refer to the same component.

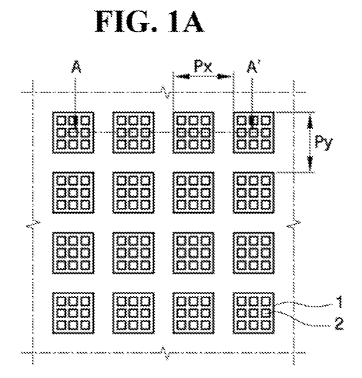

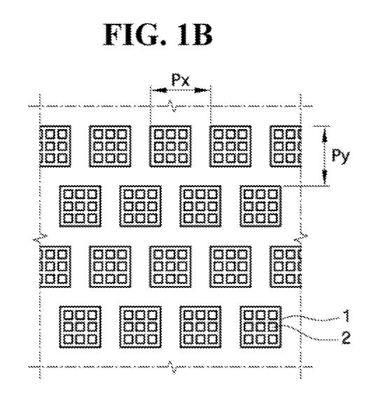

FIGS. 1A and 1B are layouts of probe cell active regions of oligomer probe arrays according to at least one embodiment of the present invention.

Referring to FIG. 1A, a plurality of probe cell active region patterns 1 are arranged in the form of a matrix comprised of rows and columns. In detail, the probe cell active region patterns 1 are arranged at a first pitch Px in the X-axis direction and at a second pitch Py in the Y-axis direction. Although FIG. 1A illustrates the first pitch Px as the same as the second pitch Py, the first pitch Px may be different from the second pitch Py when needed. A plurality of groove patterns 2 are arranged in each of the probe cell active region patterns 1. FIG. 1A illustrates groove patterns 2 having a square shape. However, it should be understood that the groove patterns 2 may have one of various shapes such as a rectangular shape, a circular shape, or a semicircular shape. The groove patterns 2 may be line patterns running in one direction across the probe cell active patterns 1 or cross-line patterns extending both in the x-axis and y-axis directions.

Referring to FIG. 1B, a plurality of probe cell active region patterns 1 are arranged at a predetermined pitch Px in the x-axis direction and at a predetermined pitch Py in the y-axis direction. Probe cell active region patterns in odd-numbered rows are offset with respect to probe cell active patterns in even-numbered rows in such a way that the probe cell active patterns in odd-numbered rows partially overlap with the probe cell active patterns in even-numbered rows. A plurality of groove patterns 2 are arranged in each of the probe cell active patterns 1.

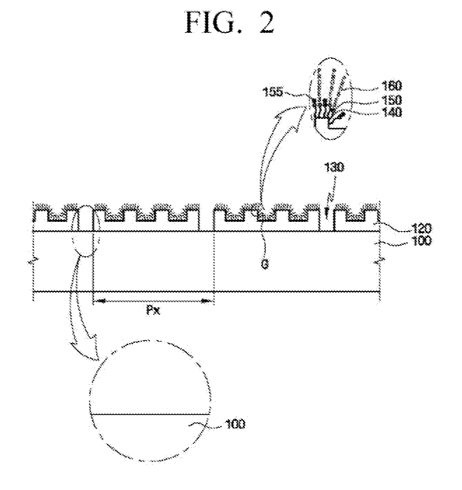

FIGS. 2 through 13 are sectional views illustrating oligomer probe arrays including probe cell active regions manufactured using the layout of FIG. 1A or 1B, according to at least one embodiment of the present invention.

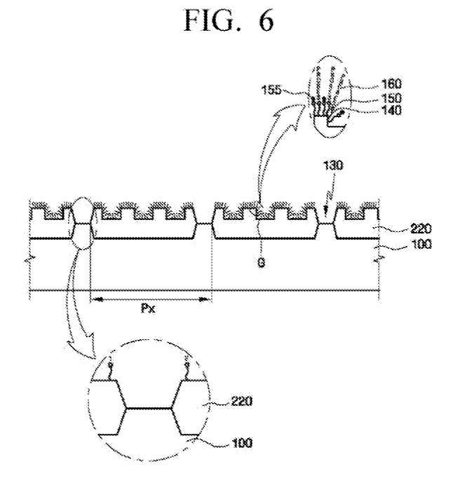

FIGS. 2 through 5 illustrate oligomer probe arrays including probe cell active regions 120 patterned on a substrate FIGS. 6 through 9 illustrate oligomer probe arrays including probe cell active regions 220 which are formed from LOCOS (LOCal Oxidation of Silicon) oxide layers formed by local oxidation of a substrate. FIGS 10 through 13 illustrate oligomer probe arrays including trench-type probe cell active regions 320 formed in the substrate.

Referring to FIGS. 2 through 13, oligomer probe arrays according to at least one embodiment of the present invention include a substrate 100, a plurality of probe cell active regions 120, 220, or 320 on or in the substrate 100, and a probe cell isolation region 130 defining a plurality of the probe cell active regions 120, 220, or 320. Each of the plurality of probe cell active regions 120, 220, or 320 have a three-dimensional surface and are coupled with at least one oligomer probe 160 with its own sequence, i.e., each one of the probe cell active regions 120, 220, or 320 is coupled to one or more oligomer probes having the same sequence, which is different from the sequences of oligomer probes coupled to the other probe cell active regions, while the probe cell isolation region 130 has no functional group for coupling with the oligomer probes 160 on its surface.

As used herein, the term “oligomer” is a low-molecular weight polymer molecule consisting of two or more covalently bound monomers. Oligomers have a molecular weight of about 1,000 or less but the present invention is not limited thereto. The oligomer may include about 2-500 monomers, preferably 5-30 monomers. The monomers may be nucleosides, nucleotides, amino acids, peptides, etc. according to the type of probes, in the present invention, previously synthesized oligomer probes may be coupled to active regions, or oligomer probes may be synthesized on active regions by in-situ photolithography.

As used herein, the terms “nucleosides” and “nucleotides” include not only known purine and pyrimidine bases, but also methylated purines or pyrimidines, acylated purines or pyrimidines, etc. Furthermore, the “nucleosides” and “nucleotides” include not only known (deoxy)ribose, but also a modified sugar wherein one or more of hydroxyl groups are replaced with halogen atoms or aliphatic groups or are functionalized as ethers, amines or the like. As used herein, the term “amino acids” are intended to refer to not only naturally occurring, L-, D-, or nonchiral amino acids, but also modified amino acids, amino acid analogs, etc.

As used herein, the term “peptides” refers to compounds produced by an amide formation between the earhoxyl group of one amino acid and the amino group of another amino acid.

The substrate 100 may be made of a material capable of minimizing or at least substantially preventing unwanted non-specific bonds during hybridization. Furthermore, the substrate 100 may be made of a material transmitting visible and/or UV radiation. The substrate 100 may be a flexible or rigid substrate. When a flexible substrate is used as the substrate 100, the substrate 100 may be a nylon membrane, a nitrocellulose membrane, a plastic film, etc. When a rigid substrate is used as the substrate 100, the substrate 100 may be a silicone substrate, a transparent glass (e.g., soda-lime glass) substrate, etc. The use of a silicone substrate or a transparent glass substrate as the substrate 100 is useful in that non-specific binding rarely occurs during hybridization. Furthermore, a transparent glass substrate is transparent to visible light and/or UV light, and thus, is useful in detection of a fluorescent material. In addition, when a silicone substrate or a transparent glass substrate is used as the substrate 100, it is possible to employ various thin layer formation processes and photolithography processes that have been well established and stably applied in the fabrication of semiconductor devices or liquid crystal display (LCD) panels.

The probe cell active regions 120,220, and 320 may be made of a material that Is substantially stable against hydrolysis upon hybridization assays, e.g., upon contacting with a pB 6-9 phosphate or Tris buffer. Thus, the probe cell active regions 120, 220, and 320 may be made of a silicon oxide layer such as a PE-TEOS layer, a HDP oxide layer, a P—SiH₄ oxide layer or a thermal oxide layer; silicate such as hafnium silicate or zirconium silicate; a silicon nitride layer; a silicon oxynitride layer; a metallic oxynitride layer such as a hafnium oxynitride layer or a zirconium oxynitride layer; a metal oxide layer such as ITO; a metal such as gold, silver, copper or palladium; polyimide; polyamine, or polymers such as polystyrene or polyacrylate. With a view to the fabrication process, the probe cell active regions 120, 220, and 320 may be made of a material that has been stably applied in the fabrication of semiconductors or LCDs

In the oligomer probe arrays shown in FIGS. 2 through 13, functional groups 150 capable of directly or indirectly coupling with the oligomer probes 160 or monomers for in-situ synthesis of the oligomer probes 160 (hereinafter, simply referred to as “functional groups 150 capable of coupling with the oligomer probes 160”) are present on surfaces of the probe cell active regions 120, 220, and 320, but absent on a surface of the probe cell isolation region 130.

The functional groups 150 are groups that can be used as starting points for organic synthesis. That is, the functional groups 150 are groups capable of directly or indirectly coupling with, e.g., covalently or non-covalently binding with, the previously synthesized oligomer probes 160 or the monomers (e.g., nucleosides, nucleotides, amino acids, or peptides) for in-situ synthesis of the oligomer probes 160. Indirect coupling may mean coupling using interposed linker.

The functional groups 150 are not limited to any particular functional groups, provided that they can be coupled to the oligomer probes 160 or the monomers for in-situ synthesis of the oligomer probes 160. Examples of the functional groups 150 include hydroxyl groups, aldehyde groups, carboxyl groups, amino groups, amide groups, thiol groups, halo groups, and sulfonate groups.

Thus, the oligomer probes 160 are coupled to the probe cell active regions 120, 220, and 320 but not to the probe cell isolation region 130 surrounding the probe cell active regions 120, 220, and 320. Therefore, a SNR can be increased in oligomer probe array-based analyses, thereby increasing analysis accuracy.

FIGS. 2 through 13 illustrate that the functional groups 150 capable of coupling with, e.g., covalently binding with, the oligomer probes 160, are connected to the surfaces of the probe cell active regions 120, 220, and 320 via linkers 140.

However, in a case where a material comprising the probe cell active regions 120, 220, and 320 includes the functional groups 150, the linkers 140 may be omitted. Even in a case where the functional groups 150 are not included in a material constituting the probe cell active regions 120, 220, and 320, they can be directly provided on the surfaces of the probe cell active regions 120, 220, and 320 by a surface treatment. The surface treatment may be ozonolysis, acid treatment, base treatment, etc. That is, the formation of the linkers 140 is optional.

The linkers 140, when used, serve to facilitate free interaction (e.g., hybridization) between the oligomer probes 160 and a target sample. Thus, the linkers 140 may have a sufficient length to ensure free probe-target, interaction. The molecular length of the linkers 140 may be 6-50 atoms, but an embodiment of the invention is not limited thereto. Two or more interconnected linkers may also be used.

The linkers 140 may be made of a material including coupling groups capable of coupling with the probe cell active regions 120, 220, and 320 and the functional groups 150 capable of coupling with monomers for in-situ synthesis of the oligomer probes 160. The functional groups 150 may be protected with protecting groups. Furthermore, protecting group may be attached to the linkers 140 coupled to the probe cell active regions 120, 220, and 320, before the in-situ synthesis of the oligomer probes 160 is carried out. Protecting groups prevent the site to which they are attached from participating in the chemical reaction to be carried out. Deprotection refers to the removal of the protecting groups to render the inactivated moieties chemically reactive. For example, acid-labile or photolabile protecting groups may be attached to the functional groups 150 of the linkers 140 to protect the functional groups 150 and then the protecting groups may be removed to expose the functional groups 150 before monomers used for in-situ photolithographic synthesis or the synthetic oligomer probes 160 are coupled to the probe cell active regions 120, 220, and 320,

When the probe cell active regions 120, 220, and 320 are made of silicon oxide, silicate, or silicon oxynitride, the coupling groups of the linkers 140 may include silicone groups capable of producing siloxane (Si—O) bonds with Si(OH) groups on surfaces of the probe cell active regions 120, 220, and 320, for example, —Si(OMe)₃. —SiMe(OMe)₂, —SiMeCl₂, —SiMe(OEt)₂, —SiCl₃, —Si(OEt)₃, and the like. Examples of the material including the functional group 150 and containing a silicon group capable of creating a siloxane bond include N-(3-(triethoxysilyl)-propyl)-4-hydroxybutyramide, N, N-bis(hydroxyethyl) aminopropyl-triethoxysilane, acetoxypropyl-triethoxysilane, 3-glycidoxy propyltrimethoxysilane, silicone compounds disclosed in International Patent Publication No. WO 00/21967, the contents of which are hereby incorporated by reference In their entirety.

When the probe cell active regions 120, 220, and 320 are made of metal oxide, the coupling groups of the linkers 140 may include metal alkoxide groups or metal carboxylate groups.

When the probe cell active regions 120, 220, and 320 are made of silicon nitride, silicon oxynitride, metal oxynitride, polyimide, or poly amine, the coupling groups of the linkers 140 may include anhydride groups, acid chloride groups, alkyl halide groups, or chlorocarbonate groups.

When the probe cell active regions 120, 220, and 320 are made of metal, the coupling groups of the linkers 140 may include sulfide groups, selenide groups, arsenide groups, telluride groups, or antimonide groups.

When the probe cell active regions 120, 220, and 320 are made of a polymer, the coupling groups of the linkers 140 may include acrylic groups, styryl groups, or vinyl groups.

The probe cell active regions 120, 220, and 320 have a three-dimensional surface. Thus, an area capable of coupling with the oligomer probes 160 can be increased, and thus, the number of the oligomer probes 160 coupled to the probe cell active regions 120, 220, and 320 can be increased, compared to conventional oligomer probe arrays having the same design rule as the oligomer probe arrays of the present invention. Therefore, even when a reduced design rule is employed, desired detection sensitivity can be ensured.

As used herein, the term “three-dimensional surface” refers to a three-dimensional surface structure of the probe cell active regions 120, 220, and 320 that is defined by one or more grooves G formed in the probe cell active regions 120, 220, and 320. However, it should be understood that structures capable of defining a three-dimensional surface are not limited to the grooves G.

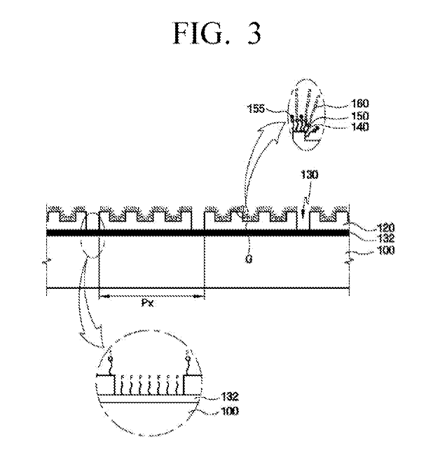

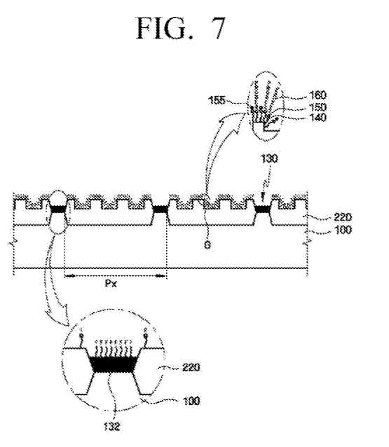

The functional groups 150 for coupling with the oligomer probes 160 are absent on the surface of the probe cell isolation region 130. In detail, according to at least one embodiment of the invention, the probe cell isolation region 130 may be an exposed surface region of a silicone substrate or a transparent substrate (see FIGS. 2, 6 and 10). According to at least one embodiment of the invention, the probe cell isolation region 130 may be a blocking layer 132 formed on the entire surface of the substrate 100 and exposed by the probe cell active region 120 (see FIG. 3), or a blocking layer 132 formed on an exposed region of the substrate 100 through the probe cell active regions 220 and 320 (see FIGS. 7 and 11). The blocking layers 132 may be made of fluorine-containing material such as fluorosilane. Also, the blocking layers 132 may be silicide layers, polysilicone layers, or epitaxial layers of Si or SiGe.

In other embodiment of the invention, the probe cell isolation region 130 may be a filler 134 that has characteristics preventing the coupling of the oligomer probes 160 and is filled into an area defined between the probe cell active regions 120, 220, and 320(see FIGS. 4, 8 and 12). The filler 134 may also be made of fluorine-containing fluoride, polysilicone, etc.

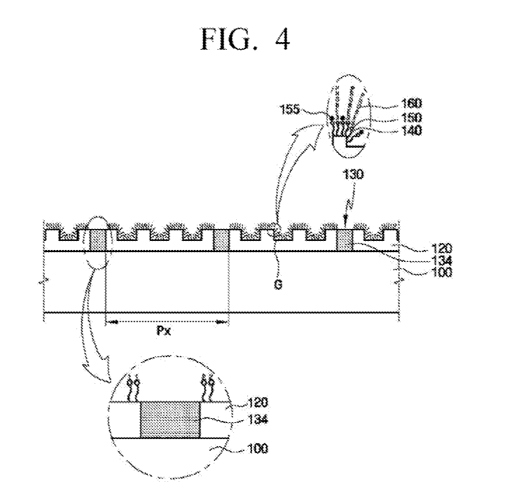

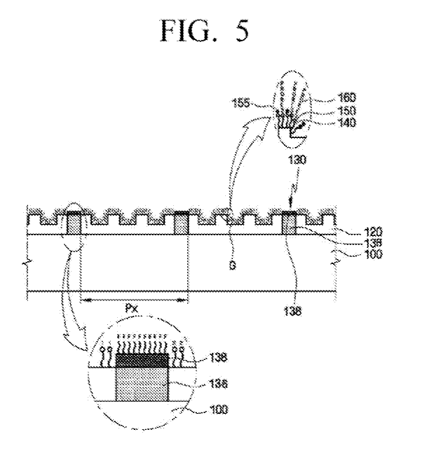

In a further embodiment of the invention, the probe cell isolation region 130 may be comprised of a filler 136 filled into an area defined between the probe cell active regions 120, 220, and 320 and a coupling blocking layer 138 formed on the filler 136(see FIGS. 5, 9, and 13). In this case, it is not necessarily required that, the filler 136 has characteristics preventing the coupling of the oligomer probes 160.

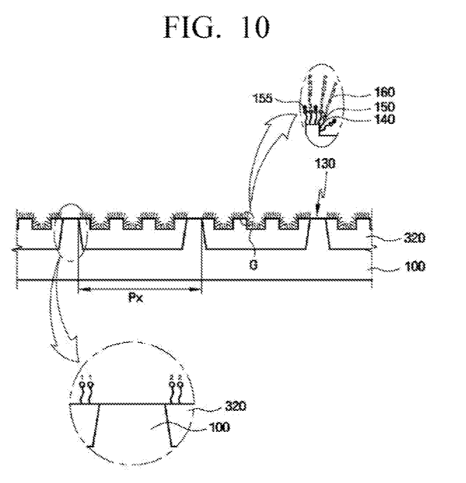

Hereinafter, methods of manufacturing oligomer probe arrays according to at least one embodiment of the present invention will be described with reference to FIGS. 14 through 29.

FIGS. 14 through 1.7 are sectional views of intermediate structures illustrating a method of manufacturing an oligomer probe array as illustrated in FIG. 2 according to an embodiment of the present invention.

Referring to FIG. 14, first, a probe cell active layer 120a is formed on a substrate 100. The probe cell active layer 120a is preferably made of a silicon oxide layer such as a PE-TEOS layer, a HDP oxide layer, a P—SiH₄ oxide layer or a thermal oxide layer; silicate such as hafnium silicate or zirconium silicate; a silicon nitride layer; a silicon oxynitride layer; a metallic oxynitride layer such as a hafnium oxynitride layer or a zirconium oxynitride layer; a metal oxide layer such as ITO, a metal such as gold, silver, copper or palladium; polyimide; polyamine; or polymers such as polystyrene or polyacrylate. The formation of the probe cell active layer 120a may be performed using a deposition method that has been stably applied in a semiconductor or LCD fabrication process, e.g., CVD (Chemical Vapor Deposition), SACVD (Sub-Atmospheric CVD), LPCVD (Low Pressure CVD), PECVD (Plasma Enhanced CVD), sputtering, or spin-coating. The probe cell active layer 120a may be formed using a material capable of being stably deposited on the substrate 100. Then, a photoresist layer PRa is formed on the probe cell active layer 120a, and then exposed to light in a projection exposure apparatus using a mask 400 manufactured according to the layout illustrated in FIG. 1A or 1B. The mask 400 may be a checkerboard type mask comprised of a transparent substrate 410 and light-shielding patterns 420, which are formed on the transparent substrate 410 and define probe cell active regions. The shapes of the light-shielding patterns 420 may vary according to the type of the photoresist layer PRa

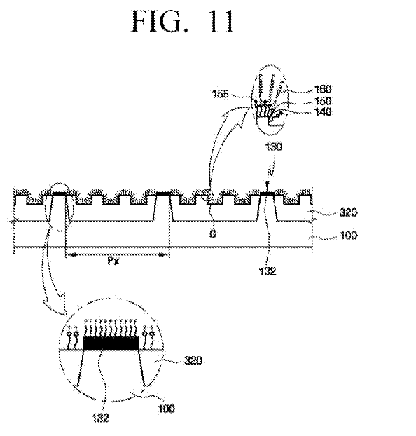

Referring to FIG. 15, the exposed photoresist layer PRa is developed to form photoresist patterns PR. Then, the probe cell active layer 120a is etched using the photoresist patterns PR as an etching mask to form probe cell active layer patterns 120b

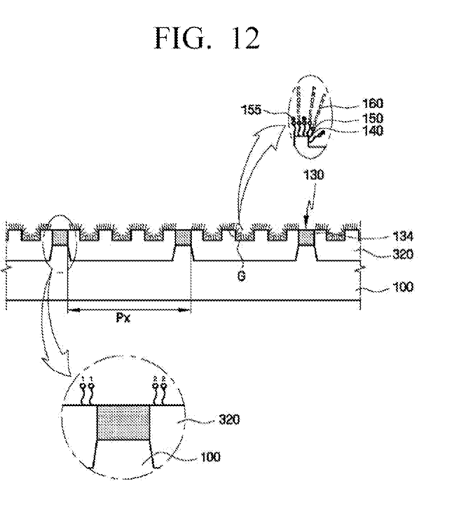

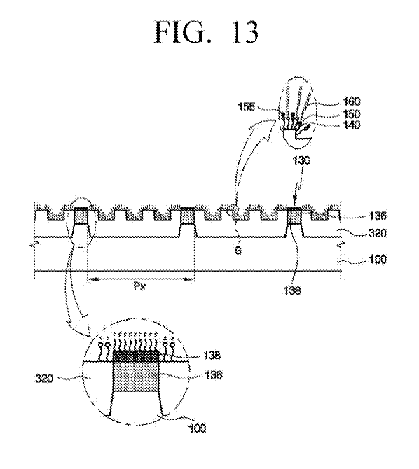

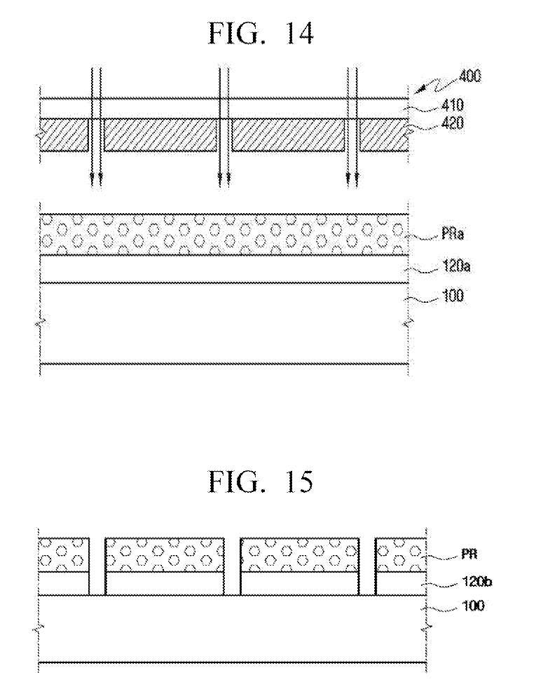

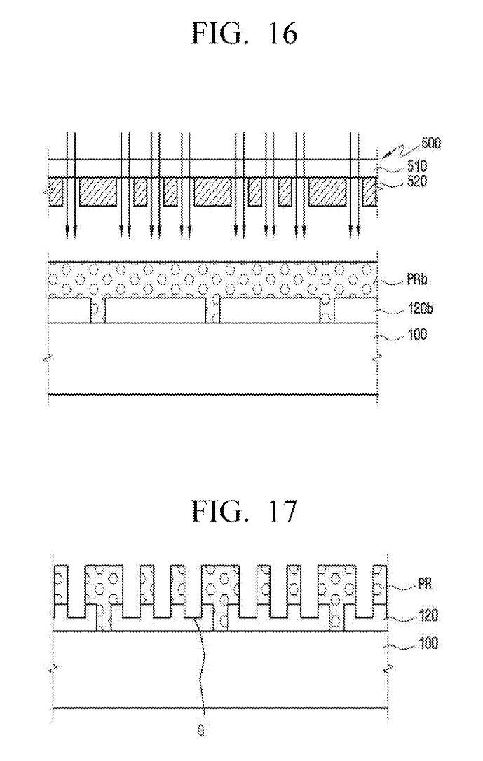

Referring to FIG. 16, the photoresist patterns PR are removed and a photoresist layer PRb is coated on the resultant structure. The photoresist layer PRb is exposed to light in a projection exposure apparatus using a mask 500 manufactured according to the groove pattern layout illustrated in FIG. 1A or 1B.

Referring to FIG. 17 the exposed photoresist layer PRb is developed to form photoresist patterns PR defining groove patterns. The probe cell active layer patterns 120b are etched using the photoresist patterns PR as an etching mask, which completes the probe cell active regions 120 including grooves G defining a three-dimensional surface.

Although not shown, formation of functional group-containing linkers on probe cell active regions made of silicon oxide will be described. SiOH groups capable of coupling with oligomer probes are exposed on surfaces of probe cell active regions made of silicon oxide. In a case where it is necessary to incorporate functional groups having better reactivity with the oligomer probes than the SiOH groups of the probe cell active regions, first linkers that can be coupled to the probe cell active regions but not to a surface of a substrate are formed on surfaces of the probe cell active regions. For example, the first linkers may have COH groups having better reactivity with the oligomer probes than the SiOH groups.

Next, second linkers having photolabile protecting groups are attached to the COH groups of the first linkers. The second linkers may be made of a material allowing the second linkers to have a sufficient length to freely interact with a target sample. Thus, the second linkers may be made of phosphoramidite having photolabile protecting groups. The photolabile protecting groups may be selected among a variety of positive photolabile groups containing nitro aromatic compounds such as o-nitrobenzyl derivatives or benzyl sulfonyl group. Exemplary examples of the photolabile protecting group include 6-nitroveratryloxycarbonyl group (NVOC), 2-nitrobenzyloxycarbonyl group (NBOC), α,α-dimethyl-dimethoxybenzyloxycarbonyl (DDZ), and the like.

Next, functional groups, i.e., the SiOH and COH groups that remain unreacted with the second linkers to be exposed to surface, are inactivated by capping to prevent the unreacted functional groups from producing noise in the oligomer probe. The capping can be performed using capping groups (see 155 of FIG. 2) capable of acetylating the SiOH and COH groups. This completes linkers (see 140 of FIG. 2) comprised of the first linkers and the second linkers in which functional groups capable of coupling with oligomer probes are protected with the photolabile protecting groups.

Next, the photolabile protecting groups of the second linkers are deprotected using a mask by exposing predetermined probe cell active regions for in-situ synthesis of oligomer probes. As a result, the functional groups (see 150 of FIG. 2) of the second linkers are exposed.

Next, the exposed functional groups are coupled with desired oligomer probes (see 160 of FIG. 2). In the case of synthesizing oligonucleotide probes using in-situ photolithography, the steps of coupling nucleotide phosphoramidite monomers having a photolabile protecting group attached thereto with the exposed functional groups 150, capping the unreacted functional groups to inactivate, and oxidation of phosphite triester structures between phosphoramidites and 5′-hydroxyl groups to phosphate triester structures are performed sequentially. Hereafter, the steps of deprotection of predetermined probe cell active regions, coupling of predetermined monomers to the probe cell active regions, capping of unreacted functional groups, and oxidation of phosphite structures to phosphate structures are sequentially repeated as described above, oligonucleotide probes having a predetermined sequence can be synthesized in each one of the probe cell active regions.

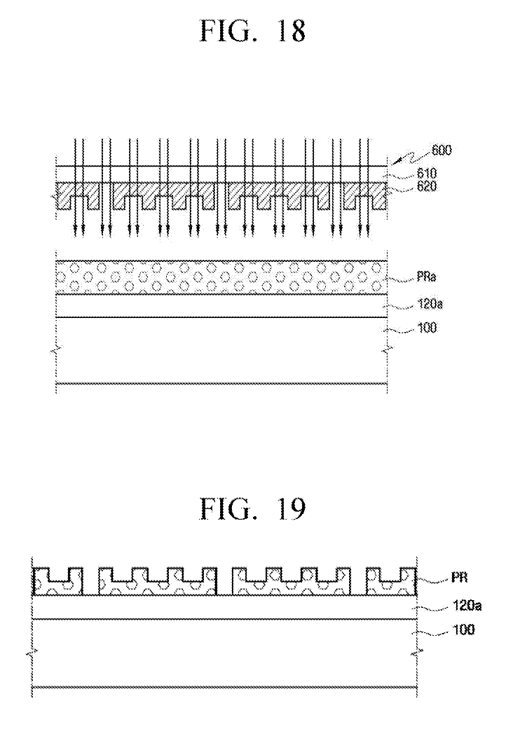

FIGS. 18 and 19 are sectional views of intermediate structures illustrating another method of manufacturing an oligomer probe array as illustrated in FIG. 2.

Referring to FIG. 18, first, a probe cell active layer 120a and a photoresist layer PRa are sequentially formed on a substrate 100. Then, the photoresist layer PRa is exposed to light using a mask 600 including a transparent substrate 610 and translucent patterns 620, which are formed on the transparent substrate 610 and have both the active patterns and the groove patterns illustrated in the layout of FIG. 1A or 1B.

Referring to FIG. 19, the exposed photoresist layer PRa is developed to form photoresist patterns PR having a three-dimensional surface.

Next, although not shown, the probe cell active layer 120a is etched using the photoresist patterns PR as an etching mask to form probe cell active regions (see 120 of FIG. 2) including grooves (see G of FIG. 2) defining a three-dimensional surface.

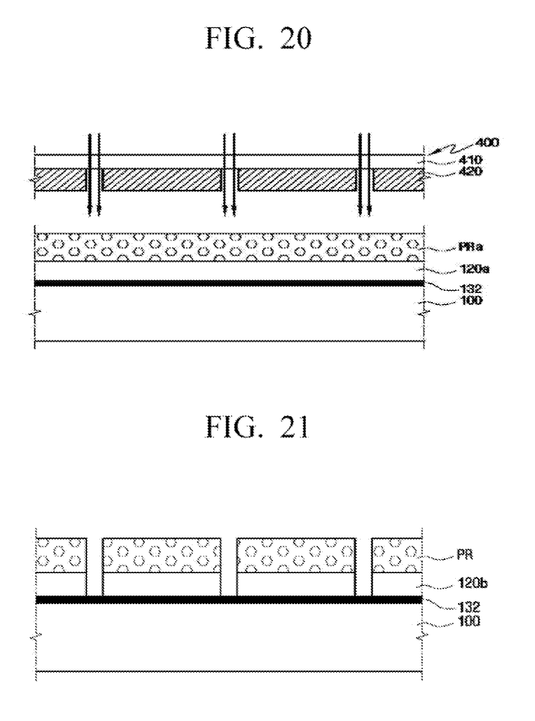

FIGS. 20 through 23 are sectional views of intermediate structures illustrating a method of manufacturing an oligomer probe array as illustrated in FIG. 3.

Referring to FIG. 20, a blocking layer 132, a probe cell active layer 120a, and a photoresist layer PRa are sequentially formed on a substrate 100. The blocking layer 132 may be a layer made of fluorine-containing fluoride, e.g., a fluorosilane layer, a silicide layer, a polysilicone layer, or an epitaxial layer of Si or SiGe.

Next, the photoresist layer PRa is exposed to light in a projection exposure apparatus using a mask 400 manufactured according to the layout illustrated in FIG. 1A or 1B.

Next, referring to FIG. 21, the exposed photoresist layer PRa is developed to form photoresist patterns PR. Then, the probe cell active layer 120a is etched using the photoresist patterns PR as an etching mask to form probe cell active layer patterns 120b. At the same time, the blocking layer 132 is partially exposed through the probe cell active layer patterns 120b to define a probe cell isolation region (see 130 of FIG. 3).

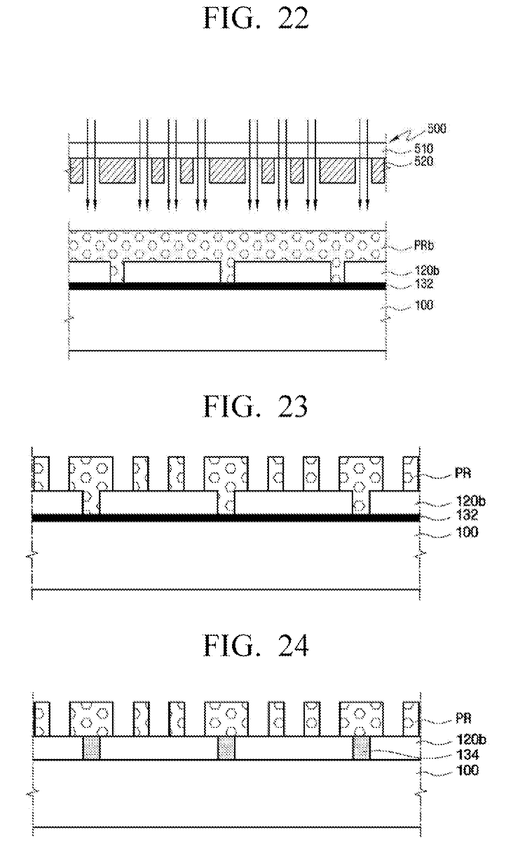

Next, referring to FIG. 22, a photoresist layer PRb is formed on the entire surface of the resultant structure and then exposed to light in a projection exposure apparatus using a mask 500 manufactured according to the groove pattern layout illustrated in FIG. 1A or 1B.

Next, referring to FIG. 23, the exposed photoresist layer PRb is developed to form photoresist patterns PR defining groove patterns.

Next, although not shown, the probe cell active layer patterns 120b are etched using the photoresist patterns PR as an etching mask to complete probe cell active regions (see 120 of FIG. 3) Including grooves (see G of FIG. 3) defining a three-dimensional surface.

FIG. 24 Is a sectional view of an intermediate structure illustrating a method of manufacturing of oligomer probe array as illustrated in FIG 4.

Referring to FIG. 24, as described above with reference to FIGS. 14 and 15, probe cell active layer patterns 120b are formed, and a filler layer (not shown) covering the resultant structure and 1filling an area defined between the probe cell active layer patterns 120b is then formed. The filler layer may be made of a material having characteristics preventing the coupling of oligomer probes and good gap-filling characteristics, e.g., fluorosilane or polysilicone.

Next, the filler layer is planarized by a Chemical Mechanical Polishing (CMP) or etch-back process to expose surfaces of the probe cell active layer patterns 120b, thereby forming a filler 134 which is filled into the area defined between the probe cell active layer patterns 120b that prevents the coupling of oligomer probes.

Next, photoresist patterns PR defining groove patterns are formed in substantially the same manner as described above with reference to FIGS. 16 and 17. Then, although not shown, the probe cell active layer patterns 120b are etched using the photoresist patterns PR as an etching mask, which completes the probe cell active regions (see 120 of FIG. 4) including grooves (see G of FIG. 4) defining a three-dimensional surface.

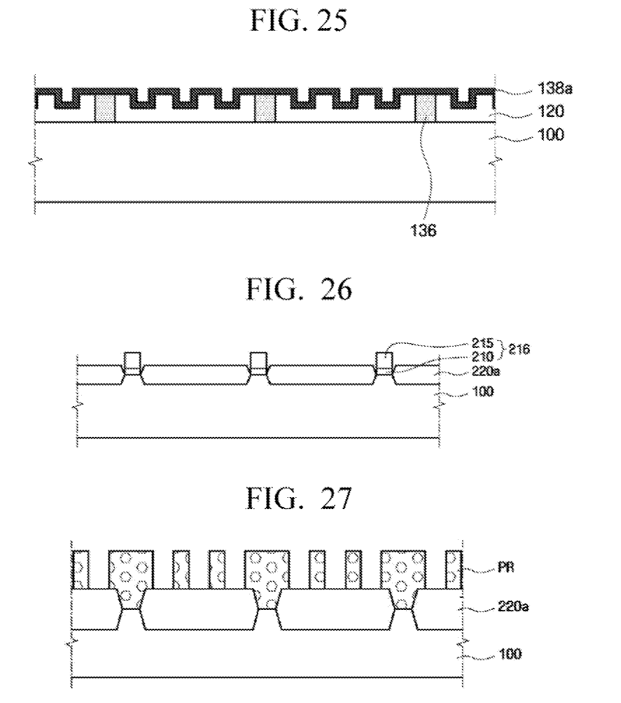

FIG. 25 is a sectional view of an intermediate structure illustrating a method of manufacturing an oligomer probe array as illustrated in FIG. 5.

Referring to FIG. 25, probe cell active regions 120 having a three-dimensional surface and a filler 136 filled into an area defined between the probe cell active regions 120 are formed on a substrate 100 in substantially the same manner as described above with reference to FIG. 24. Then, a coupling blocking layer 138a is formed on the entire surface of the substrate 100, and a portion of the blocking layer on the probe cell active regions 120 is selectively removed, which results in completion of a blocking layer pattern 138 formed on the filler 136, as shown in FIG 5. As described above, since the blocking layer pattern 138 is formed on the filler 136, it is not necessary to form the filler 136 using a material having characteristics preventing the coupling of oligomer probes. The filler 136 may be made of a material having good gap-filling characteristics.

In an alternative embodiment, in a case where the filler 136 is formed as a polysilicone layer or an epitaxial layer of Si or SiGe and the blocking layer is formed as a metal layer such as Co, Ni, or Ti, the blocking layer pattern 138 can remain only on the filler 136 by silicidation and then removal of unreacted metal layer portions.

FIGS. 26 and 27 are sectional views of intermediate structures illustrating a method of manufacturing an oligomer probe array as illustrated in FIG. 6

Referring to FIG. 26, an antioxidative pattern 216 comprised of a pad oxide layer pattern 210 and an antioxidative nitride layer pattern 215 is formed on a substrate 100. Then, a portion of the substrate 100 exposed by the antioxidative pattern 216 is oxidized using an oxidation process to form LOCOS (LOCal Oxidation of Silicon) oxide layer patterns 220a.

Next, referring to FIG 27, the antioxidative pattern 216 is removed and photoresist patterns PR defining groove patterns are formed on the resultant structure in substantially the same manner as described above with reference to FIGS. 16 and 17.

Next, although not shown, the LOCOS oxide layer patterns 220a are etched using the photoresist patterns PR as an etching mask to complete probe cell active regions (see 220 of FIG. 6) including grooves (see G of FIG. 6) defining a three-dimensional surface.

Although not shown, in a case where the pad oxide layer pattern is used as a pattern having characteristics preventing the coupling of oligomer probes, after forming the LOCOS oxide layer patterns 220a, only the antioxidative nitride layer pattern 215 is removed, allowing easy formation of a cell isolation region (see 130 of FIG. 7) including a blocking layer (see 132 of FIG. 7) preventing the coupling of oligomer probes.

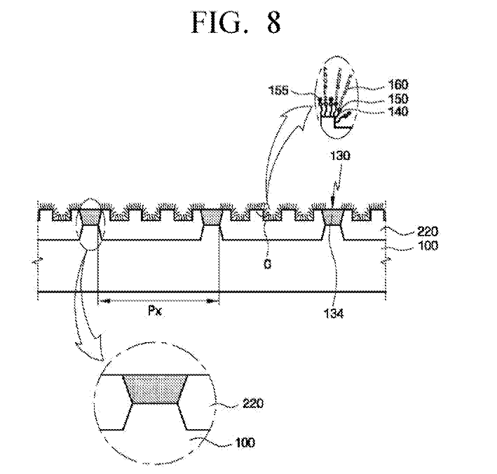

After forming LOCOS oxide layer 220a, a filler layer filling an area defined between the LOCOS oxide layer and covering the resultant structure is formed and then planarized using a CMP or etch-back process to expose surfaces of the probe cell active regions 220, thereby forming a filler (see 134 of FIG. 8) which is filled into the area defined between the probe cell active regions 220 and has characteristics preventing the coupling of oligomer probes. Then, according to substantially the same process as illustrated in FIGS. 16 and 17, photoresist patterns defining groove patterns are formed on the resultant structure and the LOCOS oxide layer 220a are then etched using the photoresist patterns as an etching mask, which results in completion of probe cell active regions (see 220 of FIG. 8) including grooves (see G of FIG. 8) defining a three-dimensional surface.

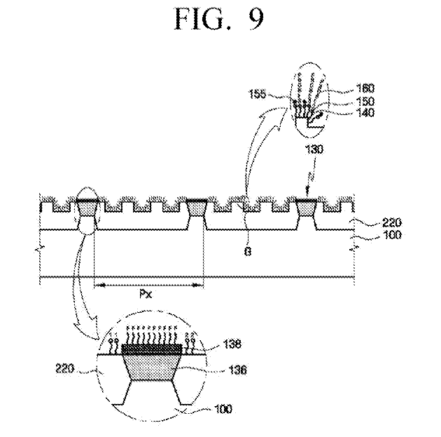

Similarly, after forming the LOCOS oxide layer 220a, a filler (see 136 of FIG. 9), which is filled into the area defined between the probe cell active regions 220 and a coupling blocking layer (see 138 of FIG. 9) formed thereon may be formed according to substantially the same process as illustrated in FIG. 25.

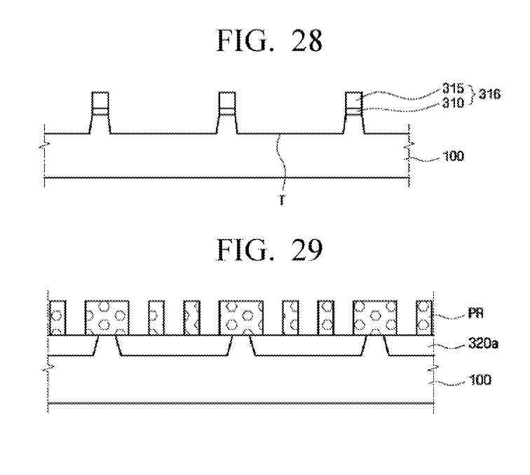

FIGS. 28 and 29 are sectional views of intermediate structures illustrating a method of manufacturing an oligomer probe array as illustrated in FIG. 10.

Referring to FIG. 28, a trench formation mask 316 comprised of a pad oxide layer 310 and a hard mask 315 defining trenches T corresponding to the active patterns illustrated in the layout of FIG. 1A or 1B is formed on a substrate 100, and the substrate 100 is etched using the trench formation mask 316 as an etching mask to form the trenches T.

Referring to FIG. 29, a probe cell active forming material as illustrated above is tilled into the trenches T and planarized by a CMP or etch-back process to form trench burial portions 320a. Then, according to substantially the same process as illustrated in FIGS. 16 and 17, photoresist patterns PR defining groove patterns are formed on the resultant structure and the trench burial portions 320a are etched using the photoresist patterns PR as an etching mask to form probe cell active regions (see 320 of FIG. 10) including grooves (see G of FIG. 10) defining a three-dimensional surface.

Although not shown, in the case of forming a blocking layer instead of the pad oxide layer 310, after forming the trench burial portions 320a, only the hard mask 315 is removed, allowing easy formation of a cell isolation region (see 130 of FIG. 11) including a blocking layer (see 132 of FIG. 11)

When the trench formation mask 316 is formed using a material having characteristics preventing the coupling of oligomer probes, and the probe cell active forming material is filled into the trenches T to reach an upper surface of the trench formation mask 316 and then planarized to expose the upper surface of the trench formation mask 316, the formation of a filler (see 134 of FIG. 12) preventing the coupling of oligomer probes can be completed in an area defined between the trench burial portions 320a through a simplified process.

Likewise, a filler (see 136 of FIG. 13) and a blocking layer (see 138 of FIG. 13) are formed by according to substantially the same process as illustrated in FIG. 25, after forming the trench burial portions 320a.

Embodiments of the invention will be described in detail through the following concrete experimental examples.

### EXPERIMENTAL EXAMPLE 1

### Manufacturing of Oligomer Probe Cell Active Regions

A PE-TEOS layer was formed to a thickness of 500 nm on silicone wafers using a CVD process. Then, a photoresist layer was formed to a thickness of 3.0 μm on the resultant structures using a spin-coating process and baked at 100° C. for 60 seconds. Then, the photoresist layer was exposed to light using a checkerboard type mask with a pitch of 1.0 μm in a 365 nm-wavelength projection exposure apparatus and developed with a 2.38% TetraMethylAmmonium Hydroxide (TMAH) solution to form checkerboard type photoresist patterns so that the underlying PE-TEOS layer was exposed in the form of a plurality of intersecting stripes. The PE-TEOS layer was etched using the photoresist patterns as an etching mask to form PE-TEOS layer patterns.

Next, a photoresist layer was formed to a thickness of 0.7 μm on the entire surface of the resultant structures using a spin-coating process and baked at 100° C. for 60 seconds. Then, the photoresist layer was exposed to light using a mask having checkerboard type grids with a pitch of 1.0 μm, each grid having 3×3 checkerboard type openings with a pitch of 300 nm, in a 248 nm-wavelength projection exposure apparatus, and then developed with a 2.38% TMAH solution to form photoresist patterns. The PE-TEOS layer patterns were etched to a depth of 300 nm using the photoresist patterns as an etching mask, which resulted in completion of oligomer probe cell active regions having a three-dimensional surface.

Next, the oligomer probe cell active regions were coated with bis(hydroxyethyl)aminopropyltriethoxysilane, treated with an acetonitrile solution containing amidite activated NNPOC-tetraethyleneglycol and tetrazole (1:1) so that, phosphoramidite protected with photolabile groups was coupled to the oligomer probe cell active regions, and then acetyl-capped, which resulted in completion of protected linker structures.

### EXPERIMENTAL EXAMPLE 2

### Manufacturing of Oligomer Probe Cell Active Regions

3,-(1,1-dihydroperfluorooctyloxy)propyltriethoxysilane was spin-coated on the entire surfaces of silicone wafers using a CVD process to form a fluorosilane layer. A PE-TEOS layer was formed to a thickness of 500 nm on the fluorosilane layer. A photoresist layer was formed to a thickness of 3.0 μm on the resultant structures using a spin-coating process and baked at 100° C. for 60 seconds. Then, the photoresist layer was exposed to light using a checkerboard type mask, with a pitch of 1.0 μm in a 365 nm-wavelength projection exposure apparatus and then developed with a 2.38% TMAH solution to form checkerboard type photoresist patterns so that the underlying PE-TEOS layer was exposed in the form of a plurality of intersecting stripes. The PE-TEOS layer was etched using the photoresist patterns as an etching mask to form PE-TEOS layer patterns exposing the underlying fluorosilane layer.

Next, a photoresist layer was formed to a thickness of 0.7 μm on the entire surfaces of the resultant structures using a spin-coating process and baked at 100° C. for 60 seconds. Then, the photoresist layer was exposed to light using a mask having checkerboard type grids with a pitch of 1.0 μm, each grid having 3×3 checkerboard type openings with a pitch of 300 nm, in a 248 nm-wavelength projection exposure apparatus, and then developed with a 2.38% TMAB solution to form photoresist patterns. The PE-TEOS layer patterns were etched to a depth of 300 nm using the photoresist patterns as an etching mask to complete oligomer probe cell active regions having a three-dimensional surface.

Next, the oligomer probe cell active regions were coated with bis(hydroxyethyl)aminopropyltriethoxysilane, treated with an acetonitrile solution containing amidite activated NNPOC-tetraethyleneglycol and tetrazole (1:1) so that phosphoramidite protected with photolabile groups was coupled to the oligomer probe cell active regions, and then acetyl-capped, which resulted in completion of protected linker structures.

### EXPERIMENTAL EXAMPLE 3

### Manufacturing of Oligomer Probe Cell Active Regions

A PE-TEOS layer was formed to a thickness of 500 nm on silicone wafers using a CVD process. Then, a photoresist layer was formed to a thickness of 3.0 μm on the resultant structures using a spin-coating process and baked at 100° C. for 60 seconds. Then, the photoresist layer was exposed to light using a checkerboard type mask with a pitch of 1.0 μm in a 365 nm-wavelength projection exposure apparatus and then developed with a 2.38% TMAH solution to form checkerboard type photoresist patterns so that the underlying PE-TEOS layer was exposed in the form of a plurality of intersecting stripes. The PE-TEOS layer was etched using the photoresist patterns as an etching mask to form PE-TEOS layer patterns. Then, polysilicone was deposited on the entire surfaces of the resultant structures using a CVD process and planarized using a CMP process to form a filler having characteristics preventing the coupling of oligomer probes and being filled into an area defined between the PE-TEOS layer patterns. Then, a photoresist layer was formed to a thickness of 0.7 μm on the entire surfaces of the resultant structures using a spin-coating process and baked at 100□ for 60 seconds. Then, the photoresist layer was exposed to light using a mask having checkerboard type grids with a pitch of 1.0 μm, each grid having 3×3 checkerboard type openings with a pitch of 300 nm, in a 248 nm-wavelength projection exposure apparatus, and then developed with a 2.38% TMAH solution to form photoresist patterns. The PE-TEOS layer patterns were etched to a depth of 300 nm using the photoresist patterns as an etching mask to complete oligomer probe cell active regions having a three-dimensional surface.

Next, the oligomer probe cell active regions were coated with bis(hydroxyethyl)aminopropyltriethoxysilane, treated with an acetonitrile solution containing amidite activated NNPOC-tetraethyleneglycol and tetrazole (1:1) so that phosphoramidite protected with photolabile groups were coupled to the oligomer probe cell active regions, and then acetyl-capped, which resulted in completion of protected linker structures.

### EXPERIMENTAL EXAMPLE 4

### In-Situ Synthesis of Oligonucleotide Probes

In-situ photolithographic synthesis of oligonucleotide probes was performed on the oligomer probe cell active regions manufactured in Experimental Examples 1-3.

That is, the oligomer probe cell active regions were exposed to light using a binary mask exposing predetermined probe cell active regions in a 365 nm-wavelength projection exposure apparatus with an energy of 1000 mJ/cm² for one minute to deprotect terminating functional groups of the linker structures. Then, the oligomer probe cell active regions were treated with an acetonitrile solution containing amidite-activated nucleotide and tetrazole (1:1) to achieve coupling of the protected nucleotide monomers to the deprotected linker structures, and then treated with a THF solution (acetic anhydride (Ac20)/pyridine (py)/methylimidazole=1:1:1) and a 0.02 M iodine-THF solution to perform capping and oxidation.

The above-described deprotection, coupling, capping, and oxidation processes were repeated to synthesize oligonucleotide probes having different sequences on the probe cell active regions such that oligonucleotide probes having the same sequence were coupled to each one of the probe cell active regions.

As described above, in oligomer probe arrays according to at least one embodiment of the invention, functional groups capable of coupling with oligomer probes are present on surfaces of probe cell active regions, but absent on a surface of a probe cell isolation region. Therefore, oligomer probes can be coupled to the probe cell active regions but not to the probe cell isolation region surrounding the probe cell active regions. A SNR can be increased in analyses using oligomer probe arrays, thereby increasing analysis accuracy.

Furthermore, since the probe cell active regions have a three-dimensional surface, an area capable of coupling with oligomer probes can be increased, and thus, the number of oligomer probes capable of coupling with each probe cell active region can be increased, compared to conventional oligomer probe arrays having the same design rule as the oligomer probe arrays according to at least one embodiment of the invention. Therefore, even when a reduced design rule is employed, desired detection sensitivity can be ensured.

While exemplary embodiments of the invention has been particularly shown and described above, it will be understood by those of ordinary skill in the art that various changes in form and details may be made therein without departing from the spirit and scope of these embodiments of the invention as defined by the following claims. Therefore, it is to be understood that the above-described embodiments have been provided only in a descriptive sense and will not be construed as placing any limitation on the scope of the invention.

What is claimed is:

1. An oligomer probe array comprising:
a substrate;
a plurality of probe cell active regions formed on or in the substrate, each of the plurality of probe cell active regions having a three-dimensional surface and being coupled with at least one oligomer probe with its own sequence; and
a probe cell isolation region defining the probe cell active regions and having no functional groups for coupling with the oligomer probes on a surface.

2. The oligomer probe array of claim 1, wherein the plurality of probe cell active regions comprise functional groups capable of coupling with the oligomer probes, and
wherein some of the functional groups are coupled to the oligomer probes and the other functional groups are inactived by capping.

3. The oligomer probe array of claim 2, wherein the functional groups are at least one group selected from the groups consisting of hydroxyl groups, aldehyde groups, carboxyl groups, amino groups, amide groups, thiol groups, halo groups, and sulfonate groups.

4. The oligomer probe array of claim 1, wherein the plurality of probe cell active regions are patterns of layers formed on the substrate, including either a LOCOS oxide layer formed by local oxidation of the substrate, or trench-type active regions filling trenches in the substrate.

5. The oligomer probe array of claim 4, wherein a surface of the probe cell isolation region is an exposed surface of a silicone substrate or a transparent substrate.

6. The oligomer probe array of claim 4, wherein a surface of the probe cell isolation region is a surface of a blocking layer that is disposed on an upper surface of the substrate and has characteristics preventing the coupling of the oligomer probes.

7. The oligomer probe array of claim 4, wherein a surface of the probe cell isolation region is a surface of a filler that Is filled into an area defined between the probe cell active regions and has characteristics preventing the coupling of the oligomer probes.

8. The oligomer probe array of claim 4, wherein a surface of the probe cell isolation region is a surface of a blocking layer that is disposed on a filler filled into an area defined between the probe cell active regions and has characteristics preventing the coupling of the oligomer probes.

9. The oligomer probe array of claim 1, wherein the oligomer probes are coupled to the probe cell active regions via linkers.

10. The oligomer probe array of claim L wherein the three-dimensional surface is achieved by one or more grooves formed in each probe cell active region.

11. A method of manufacturing an oligomer probe array, the method comprising:
providing a substrate;
forming a plurality of probe cell active regions with a three-dimensional surface on or in the substrate, the plurality of probe cell active region being defined by a probe cell isolation region without functional groups for coupling with oligomer probes; and
coupling the oligomer probes to the plurality of probe cell active regions such that each of the probe cell active regions is coupled with at least one oligmer probe with its own sequence.

12. The method of claim 11, wherein forming a plurality of probe cell active regions comprises forming the probe cell active regions to comprise functional groups capable of coupling with the oligomer probes, wherein some of the functional groups are coupled to the oligomer probe and the other functional groups are inactivated by capping.

13. The method of claim 12, wherein the functional groups are at least one group selected from the groups consisting of hydroxyl groups, aldehyde groups, carboxyl groups, amino groups, amide groups, thiol groups, halo groups, and sulfonate groups.

14. The method of claim 11, wherein the forming of a plurality of probe cell active regions comprises forming patterns of layers on the substrate, including either forming a LOCOS oxide layer by local oxidation of the substrate, or forming trench-type active regions filling trenches in the substrate.

15. The method of claim 14, wherein a surface of the probe cell isolation region is an exposed surface of a silicone substrate or a transparent substrate.

16. The method of claim 14, wherein a surface of the probe cell isolation region is a surface of a blocking layer that is disposed on an upper surface of the substrate and has characteristics preventing the coupling of the oligomer probes.

17. The method of claim 14, wherein a surface of the probe cell isolation region is a surface of a filler that is filled into an area defined between the probe cell active regions and has characteristics preventing the coupling of the oligomer probes.

18. The method of claim 14, wherein a surface of the probe cell isolation region is a surface of a blocking layer that is disposed on a filler filled into an area defined between the probe cell active regions and has characteristics preventing the coupling of the oligomer probes.

19. The method of claim 11, wherein coupling the oligomer probes to the probe cell active regions comprises coupling the oligomer probes to the probe cell active regions via linkers.

20. The method of claim 11, wherein forming the three-dimensional surface of the plurality of probe cell active regions comprises forming one or more grooves in each probe cell active region
